(19) United States
(12) Patent Application Publication
(10) Pub. No.: US 2026/0191902 A1
(43) Pub. Date: Jul. 9, 2026

(54) ZINC-BASED NANOCLUSTERS, METHODS FOR OBTAINING SAME AND USES THEREOF FOR COMBATTING ZINC DEFICIENCIES

(71) Applicants: Université de Lorraine, NANCY (FR); CENTRE HOSPITALIER REGIONAL UNIVERSITAIRE DE NANCY, NANCY (FR); INSTITUT NATIONAL DE LA SANTÉ ET DE LA RECHERCHE MÉDICALE, PARIS (FR)

(72) Inventors: Ariane BOUDIER, SAINT-MAX (FR); Igor CLAROT, EINVAUX (FR); François FEILLET, DOMBASLE SUR MEURTHE (FR)

(73) Assignees: Université de Lorraine, NANCY (FR); CENTRE HOSPITALIER REGIONAL UNIVERSITAIRE DE NANCY, NANCY (FR); INSTITUT NATIONAL DE LA SANTÉ ET DE LA RECHERCHE MÉDICALE, PARIS (FR)

(21) Appl. No.: 19/128,265

(22) PCT Filed: Nov. 9, 2023
(86) PCT No.: PCT/EP2023/081351

§ 371 Date: May 8, 2025

(30) Foreign Application Priority Data
Nov. 10, 2022 (EP) 22315282.8

(51) Int. Cl. A61K 33/30; A61K 9/14; A61K 33/04

(52) U.S. Cl. CPC A61K 33/30; A61K 9/145; A61K 33/04

(57) ABSTRACT

Zinc-based nanoclusters having: a zinc-based metal core covered over its entire surface with a mixed layer including histidine, acetate ions, and ascorbate ions, the metal core composed of zinc or a mixture of 80 to 99.99 wt % zinc and 0.01 to 20 wt % selenium; a spherical shape; a hydrodynamic diameter of 0.6 to 2.0 nm; a metal core diameter of 0.5 to 1.5 nm; a stability of 5 to 20 weeks in liquid form stored at 4° C.; a stability of at least 12 months in dry form stored at 4° C. under nitrogen; a shoulder in the UV-visible spectrum at 300±15 nm and a fluorescence spectrum with excitation wavelengths of 364±15 nm and emission wavelengths of 415±15 nm. Also, a method of preparing the zinc-based nanoclusters and their use for combating zinc deficiencies or zinc and selenium deficiencies.

### FIELD

The present invention relates to the field of chemistry, and more specifically to pharmaceutical chemistry. The invention concerns zinc-based nanoclusters, methods for obtaining them, and their uses for combating zinc deficiencies.

The zinc-based nanoclusters described in the present invention are, more particularly, nanoclusters comprising a metal core composed solely of zinc or a metal core composed of a mixture of zinc and selenium but with zinc in the clear majority within said core (from 80 to 99.99% zinc).

### BACKGROUND

Zinc is an essential trace element in the human body, which is why its deficiency can have numerous health consequences. Zinc activates more than 200 enzymes. It plays a major role in all stages of protein synthesis, it activates DNA and RNA polymerases, and it is essential for histone regulation. It is involved in the metabolism of polyunsaturated fatty acids and in the synthesis of prostaglandins. It is also involved in stabilizing the structure of certain peptide hormones (insulin, glucagon, thymulin) and plays an antioxidant role through various mechanisms. Zinc is also necessary for the metabolism of carbohydrates and lipids, and plays a major role in cell growth and in the functioning of the immune system. Lastly, zinc is also important in taste perception. Through these various mechanisms, zinc makes the body more resistant, particularly during growth and aging. In conclusion, zinc has beneficial effects on health and strengthens natural immune defenses.

Zinc deficiency is common, particularly in all malnutrition situations. It manifests as immune disorders, oligospermia, skin lesions, particularly around the orifices, hair loss, delayed healing, diarrhea, as well as vision, smell, and taste disorders leading to anorexia. In pregnant women, zinc deficiency can lead to fetal malformations or hypotrophy. Finally, in children, zinc deficiency is accompanied by delayed growth and delayed sexual maturation.

A genetic deficiency in the intestinal zinc transporter does exist: acrodermatitis enteropathica (SLC39A4 deficiency). This deficiency leads to severe and early symptoms related to zinc deficiency (cutaneous, gastrointestinal, and neurological impacts). Furthermore, all forms of malnutrition are often accompanied by zinc deficiency, whether protein-energy malnutrition in children or malnutrition related to bariatric surgery for obesity. It is therefore recommended to ensure adequate zinc intake, particularly during pregnancy and growth, as well as in the elderly or those with illnesses.

Given the lower bioavailability of zinc bound to plant proteins compared to zinc bound to animal proteins, zinc deficiency is an inherent risk in vegetarian diets, especially vegan.

Furthermore, various foods such as cola or lemonade hinder the proper absorption of zinc. Taking various medications such as a contraceptive pill or cortisone-based preparations may also result in a deficiency. It may also occur in cases of diabetes or hereditary zinc metabolism disorders.

Zinc deficiency is treated with zinc preparations/supplements taken orally. However, zinc intake may be associated with gastrointestinal problems, such as stomach aches, diarrhea, and abdominal cramps. Furthermore, zinc may also leave a metallic taste in the mouth several hours after a zinc preparation is taken orally. Finally, zinc preparations may interfere with the intestinal absorption of copper (zinc is an oral treatment for Wilson's disease) and cause transient hypocupremia.

Therefore, there is currently still a need to develop oral zinc-based preparations to combat zinc deficiency that do not have the aforementioned disadvantages.

Furthermore, in some individuals, a zinc deficiency may also be accompanied by a selenium deficiency. Selenium, like zinc, is an essential trace element for the human body and plays a key role throughout the entire body.

Most of selenium's biological functions are mediated by selenoproteins, including glutathione peroxidase, which play a key role in the body's antioxidant system. Their activities are directly linked to dietary selenium intake. Selenium is also involved in the synthesis of thyroid hormones.

The disease conventionally attributed to a severe selenium deficiency is called Keshan disease. It occurs with very low dietary intake of selenium (<10 μg/day) and leads to dilated cardiomyopathy with severe heart failure. Its description comes from a region of China where the dietary intake is very low (selenium-poor soils).

Given that selenium bioavailability is better when its intake is linked to animal protein, there is a risk of deficiency in vegetarian diets, and even more so in vegan diets. Various forms of malnutrition (including the consequences of bariatric surgery) are also at risk of selenium deficiency.

It is also important to mention the special situation of patients with hereditary metabolic diseases requiring a strict low-protein diet with the necessity of taking amino acid substitutes. In principle, these amino acid substitutes contain sufficient amounts of zinc and selenium, but their bioavailability and a probable competition for intestinal absorption mean that such patients are very often deficient in zinc and selenium.

A Belgian study published in September 2021 (Nutrients 2021, 13, 3304. https://doi.org/10.3390/nu13103304) showed that zinc and selenium deficiencies were linked to the severity of COVID-19 and the risk of death therefrom. It is noted that all patients who became critically ill or died in hospital had severe zinc and selenium deficiencies in their blood at admission. The introduction to this study emphasizes that zinc and selenium are essential trace elements for the proper functioning of the immune system, for cell signaling, and for antiviral defense.

Therefore, there is currently still a need to develop new oral preparations containing both zinc and selenium in order to combat both zinc and selenium deficiencies.

It is to the inventors' credit that they have developed zinc-based nanoclusters which are nanoclusters of zinc or nanoclusters of zinc and selenium, said nanoclusters having characteristics that are particularly suitable for combating zinc deficiencies and zinc and selenium deficiencies respectively. It is also to the inventors' credit that they have developed an original method of synthesizing zinc-based nanoclusters, namely zinc nanoclusters and zinc and selenium nanoclusters.

### SUMMARY

The present invention relates to zinc-based nanoclusters which have the following characteristics:
they comprise a zinc-based metal core covered over its entire surface with a mixed layer comprising histidine (His), acetate ions (Ac), and ascorbate ions (Asc), said metal core being composed of zinc (Zn) or a mixture of zinc and selenium (ZnSe), the amount of zinc in said mixture ranging from 80 to 99.99% and the amount of selenium in said mixture ranging from 0.01 to 20%, the percentages being percentages by weight relative to the total weight of the mixture,
they have a spherical shape,
they have a hydrodynamic diameter ranging from 0.6 to 2.0 nm, and preferably less than 1.0 nm,
they have a metal core diameter ranging from 0.5 to 1.5 nm, and preferably less than 1.0 nm,
they exhibit a stability duration ranging from 5 to 20 weeks when the nanoclusters are in liquid form and are stored at a temperature of 4° C.,
they exhibit a stability duration of at least 12 months, and preferably from 12 to 18 months, when the nanoclusters are in dry form and are stored at a temperature of 4° C. and under nitrogen,
they exhibit spectrophotometric properties, with a shoulder in the UV-Visible spectrum at 300±15 nm and a fluorescence spectrum with excitation wavelengths of 364±15 nm and emission wavelengths of 415±15 nm,

It being possible to refer to said zinc-based nanoclusters by using the formula “ZnNC@HisAcAsc” or “ZnSeNC@HisAcAsc” depending on whether the metal core is composed of zinc or a mixture of zinc and selenium.

The nanoclusters of the invention may, however, be referred to hereinafter as “nanoclusters”, “zinc-based nanoclusters”, “zinc nanoclusters”, “zinc and selenium nanoclusters”, “ZnNC nanoclusters”, “ZnNC” (“ZnNC” meaning “zinc nanocluster”), “ZnSeNC nanoclusters”, “ZnSeNC” (“ZnSeNC” meaning “zinc and selenium nanocluster”), “ZnNC@HisAcAsc nanoclusters”, “ZnNC@HisAcAsc”, “ZnSeNC@HisAcAsc nanoclusters”, or “ZnSeNC@HisAcAsc”.

The formulas “ZnNC@HisAcAsc” or “ZnSeNC@HisAcAsc” are the most explicit, since they describe that the zinc nanocluster or the zinc and selenium nanocluster comprises, on its surface, a layer comprising histidine as well as acetate ions and ascorbate ions.

The invention also relates to a method of preparing said zinc-based nanoclusters, which comprises the following steps:
reaction of zinc (II) acetate dihydrate with histidine, or reaction of zinc (II) acetate dihydrate and sodium selenite with histidine, in order to obtain a mixture of zinc (II) acetate dihydrate and histidine, or a mixture of zinc (II) acetate dihydrate, sodium selenite, and histidine, the molar ratio of histidine/zinc (II) acetate dihydrate or the molar ratio of histidine/(zinc (II) acetate dihydrate+sodium selenite) being greater than or equal to 10, preferably ranging from 10 to 200, and more preferably ranging from 100 to 200,
reaction of the mixture of zinc (II) acetate dihydrate and histidine with acid ascorbic acid, or reaction of the mixture of zinc (II) acetate dihydrate, sodium selenite, and histidine with ascorbic acid, in order to obtain a mixture of zinc (II) acetate dihydrate, histidine, and ascorbic acid, or a mixture of zinc (II) acetate dihydrate, sodium selenite, histidine, and ascorbic acid, the molar ratio of ascorbic acid/zinc (II) acetate dihydrate or the molar ratio of ascorbic acid/(zinc (II) acetate dihydrate+sodium selenite) being greater than or equal to 16, preferably ranging from 16 to 700, and more preferably ranging from 160 to 700,
collecting the zinc-based nanoclusters covered on their surface with a mixed layer comprising histidine, acetate ions, and ascorbate ions, more particularly the zinc nanoclusters or zinc and selenium nanoclusters coated on their surface with a mixed layer comprising histidine, acetate ions, and ascorbate ions.

The invention also relates to zinc-based nanoclusters as defined above, for use in combating zinc deficiency or zinc and selenium deficiency.

Finally, the invention also relates to a composition comprising the zinc-based nanoclusters of the invention, said composition being a medication, a dietary supplement, or a food composition.

The composition of the invention is further characterized in that it is in a form suitable for oral administration.

### BRIEF DESCRIPTION OF DRAWINGS

Other features, details, and advantages will become apparent upon reading the detailed description below and analyzing the attached figures.

FIG. 1 is a schematic representation of a zinc nanocluster “ZnNC@HisAcAsc” of the invention, consisting of a zinc metal core surrounded by a mixed corona comprising histidine, acetate ions, and ascorbate ions.

FIG. 2 is a high-performance liquid chromatography analysis (reversed-phase separation) of the zinc nanoclusters, showing the presence of acetate ions on the surface of the zinc metal core.

The chromatogram was obtained on previously purified fractions (size exclusion chromatography) of zinc nanoclusters. A chromatogram of a reference solution of sodium acetate was also performed.

FIG. 3 is a high-performance liquid chromatography analysis (reversed-phase separation) of zinc nanoclusters, showing the presence of histidine and ascorbate ions on the surface of the zinc metal core.

The chromatogram was obtained on previously purified fractions (size exclusion chromatography) of zinc nanoclusters. A chromatogram of a reference solution of histidine and ascorbic acid was also performed.

FIG. 4 illustrates the hydrodynamic diameter (in nanometers) of zinc nanoclusters evaluated by dynamic light scattering.

FIG. 5 illustrates the hydrodynamic diameter (in nanometers) of zinc nanoclusters evaluated by Taylor dispersion analysis.

FIG. 6 is a UV-Visible spectrum of the zinc nanoclusters.

FIG. 7 is a fluorescence spectrum of the zinc nanoclusters.

FIG. 8 is a schematic representation of a zinc and selenium nanocluster “ZnSeNC@HisAcAsc” of the invention, consisting of a zinc and selenium core surrounded by a mixed corona comprising histidine, acetate ions, and ascorbate ions.

FIG. 9 is a high-performance liquid chromatography analysis (reversed-phase separation) of the zinc and selenium nanoclusters, showing the presence of acetate ions on the surface of the zinc and selenium core.

FIG. 10 is a high-performance liquid chromatography analysis (reversed-phase separation) of the zinc and selenium nanoclusters, showing the presence of histidine and ascorbate ions on the surface of the zinc and selenium core.

FIG. 11 illustrates the hydrodynamic diameter (in nanometers) of the zinc and selenium nanoclusters, evaluated by dynamic light scattering.

FIG. 12 is a UV-Visible spectrum of the zinc and selenium nanoclusters.

FIG. 13 is a fluorescence spectrum of the zinc and selenium nanoclusters.

### DETAILED DESCRIPTION

### Zinc-Based Nanoclusters

The present invention therefore relates to zinc-based nanoclusters characterized in that they:
comprise a zinc-based metal core covered over its entire surface with a mixed layer comprising histidine (His), acetate ions (Ac), and ascorbate ions (Asc), said metal core being composed of zinc (Zn) or a mixture of zinc and selenium (ZnSe), the amount of zinc in said mixture ranging from 80 to 99.99% and the amount of selenium in said mixture ranging from 0.01 to 20%, the percentages being percentages by weight relative to the total weight of the mixture,
have a spherical shape,
have a hydrodynamic diameter ranging from 0.6 to 2.0 nm, and preferably less than 1.0 nm,
have a metal core diameter ranging from 0.5 to 1.5 nm, and preferably less than 1.0 nm,
exhibit a stability duration ranging from 5 to 20 weeks when the nanoclusters are in liquid form and are stored at a temperature of 4° C.,
exhibit a stability duration of at least 12 months, and preferably from 12 to 18 months, when the nanoclusters are in dry form and are stored at a temperature of 4° C. and under nitrogen,
exhibit spectrophotometric properties, with a shoulder in the UV-Visible spectrum at 300±15 nm and a fluorescence spectrum with excitation wavelengths of 364±15 nm and emission wavelengths of 415±15 nm,
it being possible to refer to said zinc-based nanoclusters by using the formula “ZnNC@HisAcAsc” or “ZnSeNC@HisAcAsc” depending on whether the metal core is composed of zinc or a mixture of zinc and selenium.

The zinc-based nanoclusters of the invention are metal nanoclusters. A metal nanocluster consists of the combination of several tens of atoms of a metallic element (in this case, the zinc alone or zinc mixed with selenium of the invention) with a metal core diameter that is less than or equal to 2.0 nanometers (nm).

The expression “a zinc-based metal core” in the present application means that the metal core is a zinc core or a zinc and selenium core.

Therefore, a zinc-based nanocluster may indicate a zinc nanocluster or a zinc and selenium nanocluster, it being understood that the core of said zinc and selenium nanocluster comprises an amount of zinc ranging from 80 to 99.99% by weight for an amount of selenium ranging from 0.01 to 20% by weight relative to the total weight of the core.

The nanoclusters of the invention are composed of a zinc-based metal core covered/wrapped/surrounded by a mixed layer/corona comprising histidine, acetate ions, and ascorbate ions.

The terms “corona” and “layer” may be used interchangeably throughout this application.

The term “mixed” is used to indicate that the corona or layer surrounding the zinc-based core comprises histidine, acetate ions, and ascorbate ions.

Similarly, the verbs “cover/wrap/surround” may be used interchangeably to indicate that the zinc-based core comprises, over its entire surface, a layer/corona of histidine, acetate ions, and ascorbate ions.

The zinc-based nanocluster as a whole has a spherical shape.

The formulas “ZnNC@HisAcAsc” and “ZnSeNC@HisAcAsc” within the meaning of the invention respectively denote a nanocluster composed of a zinc metal core coated with said mixed layer of histidine, acetate ions, and ascorbate ions, and a nanocluster composed of a zinc and selenium metal core coated with said mixed layer of histidine, acetate ions, and ascorbate ions.

The zinc-based nanoclusters thus advantageously comprise three ligands on the surface of the zinc-based core, namely histidine, acetate ions, and ascorbate ions. These three ligands are bound to the metal core by coordination bonds.

In particular, the mixed corona comprising histidine, acetate ions, and ascorbate ions imparts very high stability and low reactivity to the nanoclusters of the invention.

“Low reactivity” means low degradation, in particular related to oxidation (due to atmospheric oxygen for example).

The stability of the nanoclusters of the invention means that the structure and properties of the nanoclusters are maintained over time at a storage temperature of 4° C. A maintained structure means in particular that the composition of the nanocluster (metal core surrounded by the mixed layer/corona as defined above), its shape, and its diameter (metal core and hydrodynamic diameters) are preserved over time.

“Liquid form” of the zinc-based nanoclusters refers to a solution or a liquid mixture of zinc-based nanoclusters. The aforementioned stability of 5 to 20 weeks applies to zinc-based nanoclusters in liquid form when stored at a storage temperature of 4° C.

“Dry form” refers to a solid form that can be ground into powder if necessary. The aforementioned stability of 12 to 18 months applies to zinc-based nanoclusters in dry form when stored at a storage temperature of 4° C. and under nitrogen.

Depending on their form (liquid or solid), their stability duration will therefore vary.

The nanoclusters of the invention exhibit spectrophotometric properties, particularly fluorescence properties, that are characteristic of this scale, namely a metal core diameter that is less than or equal to 2 nm, which is intermediate between a molecule and a nanoparticle.

As its name suggests, the metal core diameter, or metal diameter, refers to the diameter formed solely by the zinc-based metal.

The hydrodynamic diameter takes into account the diameter of the metal core plus the layer/corona comprising histidine, acetate ions, and ascorbate ions. The hydrodynamic diameter therefore refers to the diameter of the entire zinc-based nanocluster.

According to one embodiment of the invention, the zinc-based nanoclusters have a metal core diameter and a hydrodynamic diameter which are more or less equivalent, and preferably less than 1.0 nm.

However, the metal core diameter will, of course, always be less than the hydrodynamic diameter.

The metal core diameter is evaluated by transmission electron microscopy, while the hydrodynamic diameter is evaluated by dynamic light scattering and/or Taylor dispersion analysis.

According to one advantageous embodiment of the invention, the zinc-based nanoclusters are zinc nanoclusters comprising a metal core composed of zinc, it being possible to refer to said nanoclusters by using the formula “ZnNC@HisAcAsc”.

According to another advantageous embodiment of the invention, the zinc-based nanoclusters are zinc and selenium nanoclusters comprising a metal core composed of a mixture of zinc and selenium, it being possible to refer to said nanoclusters by using the formula “ZnSeNC@HisAcAsc”.

According to one particularly advantageous embodiment of the invention, the zinc and selenium nanoclusters comprise a core composed of 99% zinc and 1% selenium.

According to yet another embodiment of the invention, the zinc-based nanoclusters are in liquid form or in dry form.

The dry form of the nanoclusters is advantageous in that it allows for easy storage, preservation, and transport.

According to yet another advantageous embodiment, the zinc-based nanoclusters of the invention are characterized in that they exhibit at least one of the following characteristics:
they are able to cross the intestinal barrier,
they exhibit good bioavailability,
they are biocompatible,
they are biodegradable,
they are able to be freeze-dried,
they are non-toxic to the human body,
they do not accumulate in organs such as the liver, spleen, kidneys, or lungs.

According to one advantageous embodiment, the zinc-based nanoclusters of the invention exhibit all of the characteristics described above.

The fact that the nanoclusters of the invention are not sequestered in said organs is due in particular to their small size (hydrodynamic diameter that is less than or equal to 2.0 nm, and preferably less than 1.0 nm). The size of the nanoclusters of the invention allows for longer circulation in the blood compared to larger compounds.

More specifically, the small size of the nanoclusters allows them to cross membranes (particularly intestinal) without passing through physiological absorption systems via a persorption phenomenon (spontaneous passage through the pores of a physiological system). This persorption phenomenon is the source of the toxicity risks of the nanoclusters, but it becomes a therapeutic modality if the quantitative aspect of nanocluster ingestion is controlled.

The nanoclusters of the invention possess surface properties that allow them to cross the intestinal barrier, which represents a significant advantage over oral zinc preparations which are often unable to cross the intestinal barrier.

“Good bioavailability” means that the orally administered nanoclusters reach the systemic circulation and are well distributed to target organs.

“Biocompatibility” means that the zinc-based nanoclusters are well accepted by the body's various organs without being toxic to these organs.

The fact that nanoclusters are biodegradable means that their degradation releases substances that are easily metabolized or eliminated by the body (zinc, selenium, histidine, acetate, and ascorbate).

According to one advantageous embodiment of the invention, it is possible to freeze-dry the nanoclusters. It is possible to freeze-dry them because they are completely stable. Freeze-drying thus makes it easy to store, preserve, and transport the nanoclusters. The stability of the nanoclusters is as defined above.

The advantageous properties of the nanoclusters of the invention are due in particular to the unique combination of their components, namely zinc, histidine, acetate ions, and ascorbate ions; or zinc, selenium, histidine, acetate ions, and ascorbate ions.

To the inventors' knowledge, zinc-based nanoclusters comprising a corona/mixed layer of histidine, acetate ions, and ascorbate ions, surrounding a metal core of zinc or zinc and selenium, and which exhibit the advantageous properties described above, have never been described before.

### Method of Preparing Zinc-Based Nanoclusters

The present invention also relates to a method of preparing zinc-based nanoclusters as defined above, characterized in that it comprises the following steps:
reaction of zinc (II) acetate dihydrate with histidine, or reaction of zinc (II) acetate dehydrate and sodium selenite with histidine, in order to obtain a mixture of zinc (II) acetate dihydrate and histidine, or a mixture of zinc (II) acetate dihydrate, sodium selenite, and histidine, the molar ratio of histidine/zinc (II) acetate dihydrate or the molar ratio of histidine/(zinc (II) acetate dihydrate+sodium selenite) being greater than or equal to 10, preferably ranging from 10 to 200, and more preferably ranging from 100 to 200,
reaction of the mixture of zinc (II) acetate dihydrate and histidine with ascorbic acid, or reaction of the mixture of zinc (II) acetate dihydrate, sodium selenite, and histidine with ascorbic acid, in order to obtain a mixture of zinc (II) acetate dihydrate, histidine, and ascorbic acid, or a mixture of zinc (II) acetate dihydrate, sodium selenite, histidine, and ascorbic acid, the molar ratio of ascorbic acid/zinc (II) acetate dihydrate or the molar ratio of ascorbic acid/(zinc (II) acetate dihydrate+sodium selenite) being greater than or equal to 16, preferably ranging from 16 to 700, and more preferably ranging from 160 to 700,
collecting the zinc-based nanoclusters, more particularly collecting the zinc nanoclusters or zinc and selenium nanoclusters.

The molar ratios as defined above, respectively:
between histidine and zinc (II) acetate dihydrate, or between histidine and zinc (II) acetate dihydrate plus sodium selenite, and
between ascorbic acid and zinc (II) acetate dihydrate, or between ascorbic acid and zinc (II) acetate dihydrate plus sodium selenite,
are important in that they allow the histidine ligands, acetate ions, and ascorbate ions to bind to the zinc-based metal core. This results in nanoclusters comprising three ligands on the surface of the zinc-based core, these three ligands being bound to the surface of the zinc-based core by coordination bonds.

Ascorbic acid is a reducing agent. The reaction of the mixture of zinc (II) acetate dihydrate (and optionally sodium selenite) and histidine with ascorbic acid is more particularly a reduction reaction of the mixture of zinc (II) acetate dihydrate (and optionally sodium selenite) and histidine with ascorbic acid. Ascorbic acid makes it possible, in particular, to obtain zinc-based nanoclusters that are free of any toxicity.

The present invention results in particular from the inventors' unexpected discovery that the original combination of the reagents used, namely zinc (II) acetate dihydrate, sodium selenite, histidine, and ascorbic acid, and in the proportions as defined above, allows obtaining zinc-based nanoclusters with particularly advantageous properties.

The excellent stability of the nanoclusters of the invention is one example of this. According to one embodiment of the invention, the zinc-based nanoclusters may be prepared more particularly according to the “solution-phase” protocol or according to the “solid-phase” protocol. Each of these two synthesis routes is in accordance with the method described above.

### 1/Solution-Phase Protocol

According to one advantageous embodiment of the invention, the method of preparation as defined above is more particularly characterized in that it is carried out under inert gas and in that:
the zinc (II) acetate dihydrate, sodium selenite is in solution form and the histidine is in powder form,
a solution of zinc (II) acetate dihydrate and histidine, or a solution of zinc (II) acetate dihydrate, sodium selenite, and histidine, is prepared by adding histidine to the solution of zinc (II) acetate dihydrate, or to the solution of zinc (II) acetate dihydrate and sodium selenite,
the solution of zinc (II) acetate dihydrate and histidine, or the solution of zinc (II) acetate dihydrate, sodium selenite, and histidine, is adjusted to a pH value ranging from 11 to 13, and preferably 12,
the ascorbic acid is in powder form,
a solution of zinc (II) acetate dihydrate, histidine, and ascorbic acid, or a solution of zinc (II) acetate dihydrate, sodium selenite, histidine, and ascorbic acid, is prepared by adding ascorbic acid to the solution of zinc (II) acetate dihydrate and histidine, or to the solution of zinc (II) acetate dihydrate, sodium selenite, and histidine, for which the pH has been adjusted to the above-mentioned values,
the solution of zinc (II) acetate dihydrate, histidine, and ascorbic acid, or the solution of zinc (II) acetate dihydrate, sodium selenite, histidine, and ascorbic acid, is stirred for 2 to 6 hours, and preferably 4 hours, at a temperature ranging from 35 to 45° C., and preferably 40° C.,
a solution comprising zinc-based nanoclusters is obtained at the end of the stirring step, namely a solution comprising zinc nanoclusters or zinc and selenium nanoclusters,
the solution comprising zinc-based nanoclusters is optionally dialyzed to obtain a purified solution of zinc-based nanoclusters, namely a purified solution of zinc nanoclusters or of zinc and selenium nanoclusters,
the solution comprising the zinc-based nanoclusters, optionally dialyzed, is optionally freeze-dried to obtain a dry form of zinc-based nanoclusters, namely a dry form of zinc nanoclusters or of zinc and selenium nanoclusters.

The solution comprising zinc-based nanoclusters, optionally dialyzed, has a stability duration of 5 to 20 weeks at a storage temperature of 4° C.

Dialysis allows removing anything not bound to the zinc-based metal core, such as excess histidine or ascorbic acid, or residual zinc or selenium that may be present in the zinc-based nanocluster solution. The layer comprising histidine, acetate ions, and ascorbate ions is bound to the zinc-based metal core by coordination bonds.

The dry form of the zinc-based nanoclusters, obtained after freeze-drying, has a stability duration of at least 12 months, and preferably 12 to 18 months, at a storage temperature of 4° C. and under nitrogen.

The dry form of the zinc-based nanoclusters may be reconstituted at any time by mixing in a reconstitution solvent such as purified water. “Reconstitute/reconstitution” refers to the simple process of mixing the dry form or lyophilizate with a solvent.

Analysis of the solution of zinc nanoclusters or of zinc and selenium nanoclusters that is obtained after reconstitution of the dry form shows that the zinc nanoclusters or zinc and selenium nanoclusters exhibit all of the properties defined above and are therefore exactly the same as those obtained directly from their method of preparation.

The solution of zinc-based nanoclusters that is obtained after reconstitution of the dry form exhibits a stability duration ranging from 5 to 12 weeks at a storage temperature of 4° C., preferably under nitrogen.

The method of preparation as defined above is further characterized in that it also comprises at least one characteristic selected from the following:
the inert gas is nitrogen,
the solution of zinc (II) acetate dihydrate is prepared by adding zinc (II) acetate dihydrate to filtered ultrapure water,
the solution of sodium selenite is prepared by adding sodium selenite to filtered ultrapure water,
the solution of zinc (II) acetate dihydrate has a concentration ranging from 0.5 to 5.0 mM,
the solution of sodium selenite has a concentration ranging from 0.07 to 140 mM,
the histidine concentration is higher than the concentration of the solution of zinc (II) acetate dihydrate,
the histidine concentration is higher than the concentration of the solution of sodium selenite,
the pH of the solution of zinc (II) acetate dihydrate and histidine, or of the solution of zinc (II) acetate dihydrate, sodium selenite, and histidine, is adjusted using sodium hydroxide,
the ascorbic acid concentration is equal to the histidine concentration,
the solution comprising the zinc nanoclusters, optionally dialyzed, has a zinc concentration ranging from 16 to 164 μg/mL,
the solution comprising the zinc and selenium nanoclusters, optionally dialyzed, has a zinc concentration ranging from 16 to 164 μg/mL and a selenium concentration ranging from 0.003 to 5.500 μg/mL,
the solution comprising the zinc-based nanoclusters, optionally dialyzed, is freeze-dried to obtain a dry form of zinc-based nanoclusters, namely a dry form of zinc nanoclusters or of zinc and selenium nanoclusters.

### 2/Solid-Phase Protocol

According to another advantageous embodiment of the invention, the method of preparing zinc-based nanoclusters as defined above is more particularly characterized in that:
the zinc (II) acetate dihydrate, sodium selenite, and histidine are each in powder form,
a powder mixture of zinc (II) acetate dihydrate and histidine, or a powder mixture of zinc (II) acetate dihydrate, sodium selenite, and histidine, is obtained by mixing together each of the powders of zinc (II) acetate dihydrate and histidine, or of zinc (II) acetate dihydrate, sodium selenite, and histidine,
the powder mixture of zinc (II) acetate dihydrate and histidine, or of zinc (II) acetate dihydrate, sodium selenite, and histidine, is ground until a powder mixture is obtained that is homogeneous in color and appearance,
the homogeneous powder mixture of zinc (II) acetate dihydrate and histidine, or of zinc (II) acetate dihydrate, sodium selenite, and histidine, is placed in a reactor,
the ascorbic acid is in powder form,
the ascorbic acid is added to the reactor comprising the homogeneous powder mixture of zinc (II) acetate dihydrate and histidine, or of zinc (II) acetate dihydrate, sodium selenite, and histidine,
the thus obtained powder mixture of zinc (II) acetate dihydrate, histidine, and ascorbic acid, or of zinc (II) acetate dihydrate, sodium selenite, histidine, and ascorbic acid, is stirred, then water is added dropwise into the reactor, said water being filtered ultrapure water,
the reactor is placed under inert gas and protected from light,
the mixture of zinc (II) acetate dihydrate, histidine, ascorbic acid, and water, or the mixture of zinc (II) acetate dihydrate, sodium selenite, histidine, ascorbic acid, and water, is left to stir in the reactor for 16 to 36 hours, and preferably 24 hours,
a liquid mixture comprising the zinc-based nanoclusters is obtained at the end of the previous stirring step, namely a liquid mixture of zinc nanoclusters or of zinc and selenium nanoclusters,
the liquid mixture comprising the zinc-based nanoclusters is optionally dialyzed to obtain a purified liquid mixture of zinc-based nanoclusters, namely a purified mixture of zinc nanoclusters or of zinc and selenium nanoclusters,
the liquid mixture comprising the zinc-based nanoclusters, optionally dialyzed, is optionally freeze-dried to obtain a dry form of zinc-based nanoclusters, namely a dry form of zinc nanoclusters or of zinc and selenium nanoclusters.

The method of preparation as defined above is further characterized in that it also comprises at least one characteristic selected from the following:
the histidine concentration is greater than the zinc (II) acetate dihydrate concentration,
the histidine concentration is greater than the sodium selenite concentration,
the ascorbic acid concentration is equal to the histidine concentration,
the water added to the reactor is filtered ultrapure water,
the inert gas is nitrogen,
the liquid mixture comprising the zinc nanoclusters, optionally dialyzed, has a zinc concentration ranging from 1370 to 13700 μg/mL,
the liquid mixture comprising the zinc and selenium nanoclusters, optionally dialyzed, has a zinc concentration ranging from 1370 to 13700 μg/mL and a selenium concentration ranging from 0.16 to 3300 μg/mL,
the liquid mixture comprising the zinc-based nanoclusters, optionally dialyzed, is freeze-dried to obtain a dry form of zinc-based nanoclusters, namely a dry form of zinc nanoclusters or of zinc and selenium nanoclusters.

According to another embodiment of the invention, the dry form of the zinc-based nanoclusters is stored under nitrogen, preferably in vials, and preferably at 4° C., it being possible to store said powder for a period of at least 12 months, and preferably from 12 to 18 months, with no change in the stability of the zinc-based nanoclusters.

Reconstitution of the nanocluster powder at the end of this period shows that the nanoclusters are the same as those obtained directly after their preparation (according to the “solution-phase” protocol or the “solid-phase” protocol). Indeed, the zinc-based nanoclusters exhibit all of the properties defined above.

### Usage of Zinc-Based Nanoclusters

The invention also relates to zinc-based nanoclusters as defined above or obtained according to the methods as defined above, for use as medication.

According to one advantageous embodiment, the invention relates to zinc-based nanoclusters as defined above or obtained according to the methods as defined above, for use in combating zinc deficiencies.

In such case, the zinc-based nanoclusters are more particularly zinc nanoclusters.

According to another advantageous embodiment, the invention relates to zinc-based nanoclusters as defined above or obtained according to the methods as defined above, for use in combating zinc and selenium deficiencies.

In such case, the zinc-based nanoclusters are more particularly zinc and selenium nanoclusters.

Another object of the invention is a composition characterized in that it comprises zinc-based nanoclusters as defined above or obtained according to the methods as defined above.

The composition of the invention more particularly comprises zinc nanoclusters or zinc and selenium nanoclusters.

The composition of the invention may be a medication, a dietary supplement, or a food composition.

The amount of zinc or the amount of zinc and selenium present in the composition of the invention determines whether it is a dietary supplement or a medication. Thus, a dietary supplement will comprise a lesser amount of zinc, or a lesser amount of zinc and selenium, than the amount of zinc or the amount of zinc and selenium present in a medication. According to one advantageous embodiment of the invention, the composition is in a form suitable for oral administration.

Zinc-based nanoclusters advantageously may be administered orally because they are able to pass through the intestinal barrier without difficulty, in particular due to their small size. According to another advantageous embodiment of the invention, the composition of the invention comprising the zinc nanoclusters comprises a lower amount of zinc than the amount of zinc usually present in a conventional oral zinc preparation, whether it is a medication or a dietary supplement.

According to yet another advantageous embodiment of the invention, the composition of the invention comprising zinc and selenium nanoclusters comprises a lower amount of zinc and selenium than the amount of zinc and selenium usually present in a conventional oral zinc and selenium preparation, whether it is a medication or a dietary supplement.

### EXAMPLES

The following examples illustrate the invention, but do not limit it in any way.

### Example 1

### 1/Preparation of Zinc Nanoclusters

This example respectively describes the two possible synthesis routes for preparing the zinc nanoclusters of the invention, namely the “solution-phase protocol” and the “solid-phase protocol”.

### 1.1/Solution-Phase Protocol

### Reagents Used:

Zinc (II) acetate dihydrate [Zn(CH₃COO)₂, 2H₂O], M=219.51 g/mol (Sigma Aldrich, Cas 5970-45-6);
L(−)-Histidine, M=155.15 g/mol (Merck, Cas 71-00-1);
Ascorbic acid, M=176.12 g/mol (Sigma Aldrich, Cas 50-81-7);
1M NaOH solution, M=40.00 g/mol (VWR, Cas 1310-73-2).

### Precautions

The synthesis is carried out under inert gas (nitrogen). The glassware is washed with aqua regia (1 volume of 65% nitric acid to 2 volumes of 37% hydrochloric acid).

Ultrapure water is used and is filtered through a 0.2 μm pore size filter.

### Preparation of a 2.5 mM Zinc Acetate Stock Solution

55.0 mg of zinc acetate is placed in a 100 mL volumetric flask. Filtered ultrapure water is added up to the fill line of the flask. A zinc acetate solution with a concentration of 2.5 mM is obtained. After complete dissolution, the zinc acetate solution is transferred into a suitable container. This solution may be stored for one month in a refrigerator at 4° C.

Synthesis of Zinc Nanoclusters Stabilized with Histidine

500 μL of the zinc acetate stock solution as prepared in the previous step is added to a round-necked flask capable of holding up to 50 mL of solution. Then, 4500 μL of filtered ultrapure water are added to the flask. The resulting zinc acetate solution is referred to as 1x.

The 1x zinc acetate solution is stirred at 130 rpm using a multi-plate shaker. 39 mg of histidine is added to the zinc acetate solution. The zinc acetate and histidine solution is stirred for 15 minutes. The solution is colorless.

After stirring, the pH of the zinc acetate and histidine solution is adjusted to 12 with 10 drops of 1M NaOH. 139 mg of ascorbic acid is added to the reaction mixture. 2 minutes is allowed for the ascorbic acid to dissolve completely. The flask (reactor) is then placed in a water bath at 40° C. with stirring (speed set to 6) for 4 hours.

The solution of zinc nanoclusters that is obtained from the synthesis is colorless. It is referred to as 1x and has a zinc concentration of 16 μg/mL. It is stored in a cool location at 4° C.

The solutions of zinc nanoclusters may be freeze-dried.

Zinc acetate solutions with concentrations ranging from 1x to 10x are prepared in order to obtain 1x to 10x solutions of zinc nanoclusters which have a zinc concentration ranging from 16 to 164 μg/mL.

For reference, a 2x zinc acetate solution is prepared by placing 1000 μL of zinc acetate stock solution in the flask and topping up to 5000 μL with filtered ultrapure water. A 4x zinc acetate solution is prepared by placing 2000 μL of zinc acetate stock solution in the flask and topping up to 5000 μL with filtered ultrapure water, etc.

The resulting 1x to 10x solutions of zinc nanoclusters are stored in a cool location at 4° C.

### Dialysis of Zinc Nanoclusters

The 1x solution of zinc nanoclusters that is obtained in the previous step is purified by dialysis. A dialysis device is prepared (X12 Float-a-lyzer G2, CE MWCO 100-500 D, Reference 1511160), and a 150 ml beaker is filled with 100 mL of filtered ultrapure water. The dialysis device is filled with filtered ultrapure water using a Pasteur pipette. The dialysis device is placed in the beaker while stirring (130 rpm). The device is left to hydrate and wash for 1 hour. The water is then replaced with a new volume of 100 mL of filtered ultrapure water. The dialysis device is emptied using a Pasteur pipette and then filled with the 1x solution of zinc nanoclusters, which is left to stir overnight (for 12 hours) at a temperature between 2 and 6° C. The resulting dialyzed 1x solution of zinc nanoclusters is transferred into a suitable container and stored at 4° C.

Dialysis does not affect the zinc concentration of the nanoclusters. Thus, the zinc concentrations of the dialyzed solutions of zinc nanoclusters are identical to those of non-dialyzed solutions.

The solutions of zinc nanoclusters, if dialyzed, may be freeze-dried.

The zinc concentrations of the dialyzed solutions of zinc nanoclusters are identical to those of non-dialyzed solutions, and range from 16 to 164 μg/mL for zinc acetate solutions ranging from 1x to 10x.

### 1.2/Solid-Phase Protocol

### Reagents Used and Precautions

The zinc (II) acetate dihydrate, L(−)-Histidine, and ascorbic acid are the same as those used in the solution-phase protocol. Sodium hydroxide is not required in the solid-phase protocol. The same precautions apply as for the solution-phase protocol.

Synthesis of Zinc Nanoclusters Stabilized with Histidine

23 mg of zinc acetate is weighed and placed inside an agate mortar. 1.7 g of histidine is then weighed. One part histidine powder to one part zinc acetate powder is added, taking care to grind the powders thoroughly with a pestle until a mixture of uniform color and appearance is obtained. This operation is repeated until all the histidine has been used.

The final mixture of the two powders should be white in color and homogeneous. The mixture of the two powders is then transferred to a 50 mL single-necked flask (NS 19/26 ground neck) using a spatula. 3 g of ascorbic acid is weighed and transferred into the flask. An olive-shaped magnetic stirrer is placed at the bottom of the flask. 5 mL of ultrapure water is filtered using a 5 mL plastic syringe and is added dropwise into the reactor. A liquid mixture is obtained.

The reactor is closed using a flip-top skirt cap (19.4 mm diameter) and is placed under nitrogen without creating excess pressure, using a latex balloon. The reactor is wrapped in aluminum foil and then stirred (200 rpm) for the duration of the reaction. A 24-hour wait is required for the reaction to complete. At the end of the reaction, the resulting product, which is in liquid form, is gray in color.

The resulting liquid comprising the zinc nanoclusters has a zinc concentration of 1370 μg/mL and is referred to as 100x.

The zinc concentration of the nanoclusters is, of course, dependent on the amount of zinc acetate used at the start of the method of the invention.

The above-described operations are repeated, to obtain zinc concentrations ranging from 1370 to 13700 μg/mL (100x to 1000x) for quantities of zinc acetate at the start of the method of the invention (solid-phase protocol) ranging from 23 mg to 230 mg.

The resulting liquid comprising the zinc nanoclusters is then transferred into a suitable plastic container (the final volume is not 5 mL but slightly larger, approximately 8.5 mL). The liquid comprising the zinc nanoclusters is either stored at 4° C. or is transferred to a freeze-dryer.

The freeze-drying is carried out in 1 mL portions, with no addition of additional reagents. After freeze-drying, the contents of the vial (which contains the zinc nanoclusters in dry form) are placed under nitrogen and stored at 4° C.

The dry form of the zinc nanoclusters can be reconstituted at any time in 1 mL of purified water. The solution of zinc nanoclusters reconstituted in this manner is stored at 4° C., preferably under nitrogen.

### 2/Preparation of Zinc and Selenium Nanoclusters

### 2.1/Solution-Phase Protocol

### Reagents Used:

The zinc (II) acetate dihydrate, L(−)-Histidine, ascorbic acid, and sodium hydroxide are the same as those used in the solution-phase protocol for preparing zinc nanoclusters.

Sodium selenite [Na₂SeO₃] M=172.948 g/mol (Sigma Aldrich, Cas 10102-18-8) is also used in this example.

The same precautions are required as those described for the solution-phase protocol for zinc nanoclusters.

### Preparation of a 2.5 mM Zinc Acetate Stock Solution

The zinc acetate stock solution is prepared as described in the solution-phase protocol paragraph for zinc nanoclusters.

### Preparation of a 0.71 mM Sodium Selenite Stock Solution

12.0 mg of sodium selenite is placed in a 100 mL volumetric flask. Filtered ultrapure water is added up to the fill line of the flask. A 0.71 mM sodium selenite solution is obtained. After complete dissolution, the sodium selenite solution is transferred into a suitable container. This solution can be stored for one month in a refrigerator at 4° C.

Synthesis of Zinc and Selenium Nanoclusters Stabilized with Histidine

For the synthesis of bimetal nanoclusters of zinc and selenium, a 1:10 dilution of the sodium selenite solution obtained in the previous step in 1 ml of water is performed (100 μL of sodium selenite stock solution+900 μL of ultrapure water). The 1:10 diluted sodium selenite solution is referred to as 0.1% selenium solution.

2000 μL of the zinc acetate stock solution as prepared in the previous step is added to a round-necked flask capable of holding up to 50 mL of solution. Then, 3000 μL of filtered ultrapure water is added to the flask. The resulting zinc acetate solution is referred to as 4x.

100 μL of diluted sodium selenite solution (0.1% selenium) is added to 2000 μL of 4x zinc acetate solution.

The molar ratio of selenium to zinc in the prepared zinc acetate and sodium selenite solution is 1 to 1000.

The zinc acetate and sodium selenite solution is mixed at 250 rpm on a multi-plate shaker for 5 minutes.

39 mg of histidine is added to the zinc acetate and sodium selenite solution. The solution of zinc acetate, sodium selenite, and histidine is stirred for 15 minutes at the same stirring speed. The solution is colorless.

After stirring for 15 minutes, the pH of the solution of zinc acetate, sodium selenite, and histidine is adjusted to 12 with 10 drops of 1M NaOH.

139 mg of ascorbic acid is added to the reaction mixture. 2 minutes is allowed for the ascorbic acid to dissolve completely. The flask (reactor) is placed in a water bath at 40° C. with stirring (speed set to 6) for 4 hours.

The solution of zinc and selenium nanoclusters that is obtained from the synthesis is colorless and has:
a zinc concentration of 65 g/mL for a 4x zinc acetate solution,
a selenium concentration of 0.1 μg/mL for a 0.1% sodium selenite solution.

In this example, the mass ratio of zinc to selenium within the bimetal core of the zinc and selenium nanoclusters is 99.9% zinc to 0.1% selenium.

The solution of zinc and selenium nanoclusters is stored in a cool location at 4° C. The solution may also be freeze-dried.

The same synthesis protocol may be used but instead adding 100 μL of a 0.71 mM sodium selenite solution that has been diluted 1:100 in water. A sodium selenite solution with 0.01% selenium is obtained.

100 μL of the diluted sodium selenite solution (0.01% selenium) is added to 2000 μL of the 4x zinc acetate solution.

The molar ratio of selenium to zinc in the zinc acetate and sodium selenite solution prepared in this manner is 1 to 10,000.

Zinc acetate solutions ranging in concentration from 1x to 10x, as well as diluted sodium selenite solutions containing 0.01% to 20% selenium, are prepared.

Zinc concentrations in the solutions of zinc and selenium nanoclusters range from 16 to 164 μg/mL for zinc acetate solutions ranging from 1x to 10x.

Selenium concentrations in the solutions of zinc and selenium nanoclusters range from 0.003 to 5.5 μg/mL for diluted sodium selenite solutions containing 0.01% to 20% selenium.

### Dialysis of Zinc and Selenium Nanoclusters

The solution of zinc and selenium nanoclusters that was obtained in the previous step may be purified by dialysis, using the same protocol as described in the “Zinc nanocluster dialysis” section of the solution-phase protocol for zinc nanoclusters.

The zinc concentrations in dialyzed solutions of zinc and selenium nanoclusters are identical to those in non-dialyzed solutions, and range from 16 to 164 g/mL for zinc acetate solutions ranging from 1x to 10x.

The selenium concentrations in dialyzed solutions of zinc and selenium nanoclusters are identical to those in non-dialyzed solutions, and range from 0.003 to 5.5 μg/mL for diluted sodium selenite solutions containing 0.01% to 20% selenium.

### 2.2/Solid-Phase Protocol

### Reagents Used and Precautions

The zinc (II) acetate dihydrate, L(−)-histidine, and ascorbic acid are the same as those used in the solution-phase protocol. Sodium hydroxide is not needed in the solid-phase protocol. Sodium selenite [Na₂SeO₃] M=172.948 g/mol (SIGMA ALDRICH, CAS 10102-18-8) is also used in this example.

The precautions to be taken are the same as for the solution-phase protocol.

Synthesis of Zinc and Selenium Nanoclusters Stabilized with Histidine

10 mg of sodium selenite is placed in an agate mortar. 1.27 g of zinc acetate is weighed. One part zinc acetate powder to one part sodium selenite powder is added, taking care to grind the powders thoroughly with a pestle until a mixture of uniform color and appearance is obtained. This process is repeated until all the zinc acetate has been used.

23 mg of the above powder mixture is weighed and then placed within an agate mortar. 1.7 g of histidine is then weighed. One part histidine powder to one part zinc acetate/sodium selenite powder is added, taking care to grind the powders thoroughly with a pestle until a mixture of uniform color and appearance is obtained. This process is repeated until all the histidine has been used.

The final mixture of the two powders should be white in color and homogeneous. The mixture of the two powders is then transferred to a 50 mL single-necked flask (NS 19/26 ground neck) using a spatula. 3 g of ascorbic acid is weighed and transferred into the flask. An olive-shaped magnetic stirrer is placed at the bottom of the flask. 5 mL of ultrapure water is filtered using a 5 mL plastic syringe and is added dropwise into the reactor. A liquid mixture is obtained. The reactor is closed using a flip-top skirt cap 19.4 mm in diameter is and placed under nitrogen without creating excess pressure, using a latex balloon. The reactor is wrapped in aluminum foil and stirred (200 rpm) for the duration of the reaction. A 24-hour wait is required for the reaction to complete. At the end of the reaction, the resulting product, which is in liquid form, is gray in color.

The resulting liquid comprising the zinc and selenium nanoclusters has a concentration of:
1370 μg/mL of zinc, corresponding to a zinc concentration also referred to as 100x,
16 μg/mL of selenium, corresponding to a selenium concentration also referred to as 1%.

The zinc and selenium concentrations of the zinc and selenium nanoclusters are, of course, dependent on the quantities of zinc acetate and sodium selenite used at the start of the method of the invention.

The operations described above are repeated, to obtain zinc and selenium nanoclusters comprising:
zinc concentrations ranging from 1370 to 13700 μg/mL, corresponding to 100x to 1000x zinc concentrations,
selenium concentrations ranging from 0.16 to 3300 μg/mL, corresponding to 0.01% to 20% selenium concentrations.

The liquid comprising the zinc and selenium nanoclusters is either stored at 4° C. or is transferred to a freeze-dryer.

The freeze-drying is carried out in 1 mL portions, with no addition of additional reagents. After freeze-drying, the contents of the vial (which contains the zinc and selenium nanoclusters in dry form) are placed under nitrogen and stored at 4° C.

The dry form of the zinc and selenium nanoclusters can be reconstituted at any time in 1 mL of purified water. The solution of zinc and selenium nanoclusters reconstituted in this manner is stored at 4° C., preferably under nitrogen.

### Example 2

### 1/Characterization of the Zinc Nanoclusters

The zinc nanoclusters obtained in Example 1, whether via the solution-phase or solid-phase protocol, are characterized in terms of their structure, size, spectrophotometric properties, and stability.

### Structure of the Zinc Nanoclusters

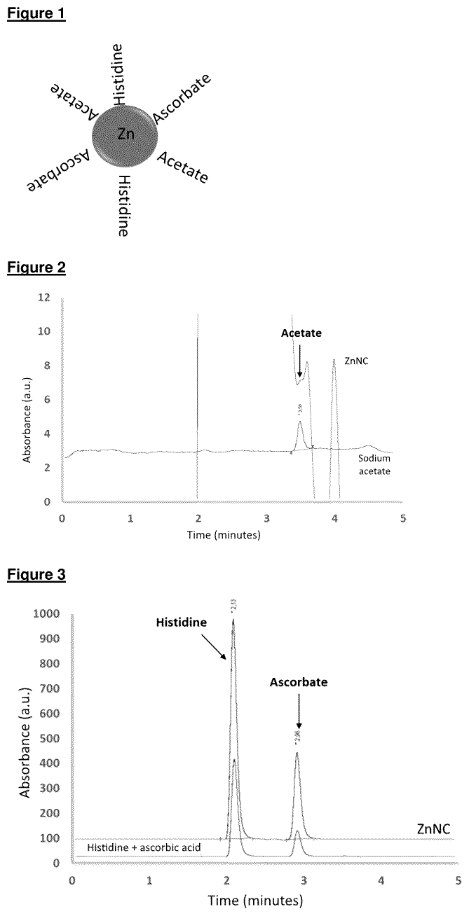

The zinc nanoclusters of the invention have, more particularly, a spherical shape. They are composed of a zinc metal core covered with a mixed corona comprising histidine, acetate ions, and ascorbate ions. FIG. 1 is a schematic representation of a zinc nanocluster of the invention, which may also be designated by the formula “ZnNC@HisAcAsc.”

The presence of acetate ions on the surface of the zinc nanoclusters was demonstrated by high-performance liquid chromatography (HPLC), more specifically by reversed-phase chromatography.

The zinc nanoclusters in the 1x dialyzed solution, which has a zinc concentration of 16 μg/mL (as obtained in Example 1, section 1.1/“Solution-phase protocol”), are destroyed (total dissolution and a return of the nanocluster structure to its various component elements) by a chemical process, namely dissolution in a concentrated acid (HCl) then in a concentrated base (NaOH), then are analyzed by reversed-phase chromatography with comparison to a sodium acetate control (ligand retention on a non-polar column, separation, identification, and quantification (external calibration) of the acetate ions by UV spectrophotometry).

The results obtained are illustrated in the chromatogram in FIG. 2. The peak at 3.58 min associated with acetate ions (control, see lower plot) is found in the zinc nanoclusters (see upper plot), thus demonstrating the presence of acetate ions on the surface of the zinc metal core.

The presence of histidine and ascorbate ions on the surface of the zinc nanoclusters was also demonstrated by high-performance liquid chromatography, more particularly after purification of the nanoclusters in solution by size exclusion chromatography, in comparison to a histidine and ascorbic acid control.

The results obtained are illustrated in the chromatogram in FIG. 3.

The peak at 2.13 min associated with histidine (control, see lower plot) is found in the zinc nanoclusters (see upper plot), thus demonstrating the presence of histidine on the surface of the metal core.

The peak at 2.96 min associated with ascorbate ions (control, see lower plot) is found in the zinc nanoclusters (see upper plot), thus demonstrating the presence of ascorbate ions on the surface of the metal core.

### Size of the Zinc Nanoclusters

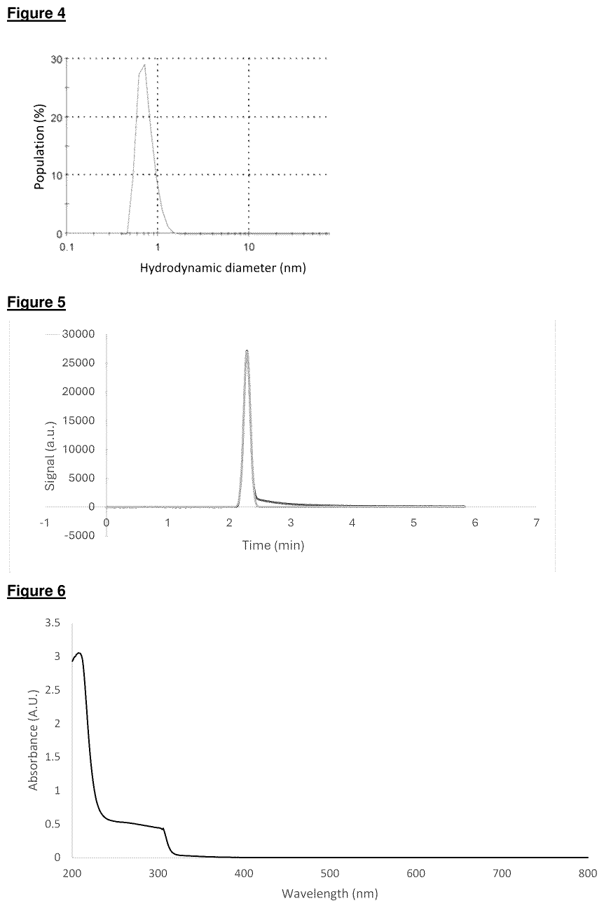

The hydrodynamic diameter (Dh) of the zinc nanoclusters was evaluated by dynamic light scattering (FIG. 4) (173° angle, 530 nm laser, 25° C. temperature in a Malvern Nanosizer) and by Taylor dispersion analysis (FIG. 5).

The method using dynamic light scattering analysis consists of analyzing the Brownian motion of particles and modeling this using the Stokes-Einstein equation.

The method using Taylor dispersion analysis involves injecting a plug of solute into an open capillary tube (50 μm) where it disperses under the influence of a hydrodynamic flow (positive pressure 1 psi, parabolic velocity profile). The principle for determining the hydrodynamic radius is based on the Taylor-Aris relationship, which establishes the link between the spread of the solute peak (modeling a Gaussian) and the molecular diffusion coefficient.

The metal core diameter of the zinc nanoclusters was evaluated by transmission electron microscopy (deposition on nickel grids, observations under beams operating at 200 kV (LaB6 cathode) Philips CM 200).

The diameters (hydrodynamic and metal core) of the zinc nanoclusters were evaluated immediately after their synthesis, for both the solution-phase and solid-phase protocols. For the solution-phase protocol, the hydrodynamic and metal core diameters were evaluated in the 1x zinc nanocluster solution, non-dialyzed and not freeze dried, obtained in section 1.1/of Example 1, which has a zinc concentration of 16 μg/mL.

For the solid-phase protocol, the hydrodynamic and metal core diameters were evaluated in the freeze-dried samples obtained in section 1.2/of Example 1, having a zinc concentration of 1370 g/mL (100x).

The average hydrodynamic diameter of the zinc nanoclusters, as with the metal core diameter, is less than 1.0 nm. More specifically, it is clear from FIGS. 4 and 5 that in dynamic light scattering (FIG. 4) and Taylor dispersion (FIG. 5), the hydrodynamic diameter of the zinc nanoclusters is equal to 0.72±0.05 nm.

### Spectrophotometric Properties

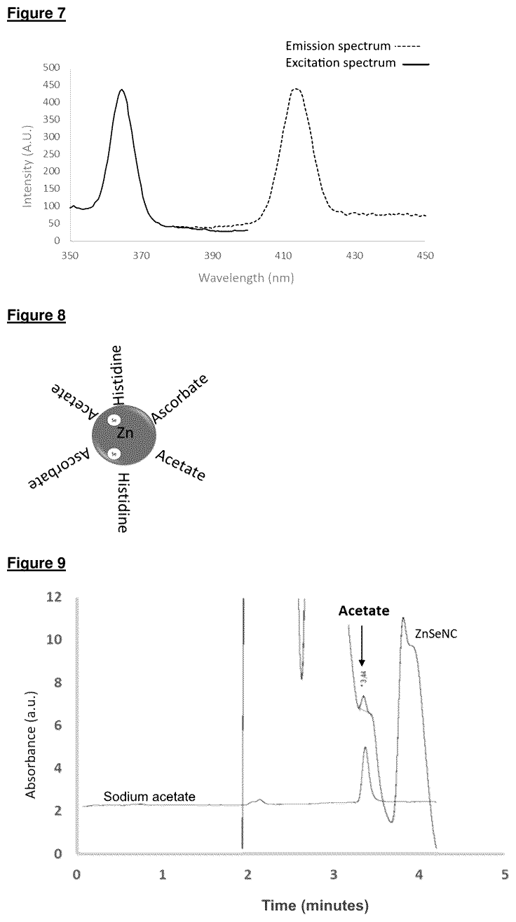

The spectrophotometric properties of the zinc nanoclusters were evaluated by UV-Visible spectroscopy (FIG. 6) and fluorescence spectroscopy (FIG. 7), immediately after their synthesis.

The UV-vis spectrum shows a shoulder at 300±15 nm (FIG. 6), confirming the existence of the nanoclusters.

The zinc nanoclusters exhibit fluorescence with an excitation wavelength of 364±15 nm and an emission wavelength of 415±15 nm (FIG. 7), also confirming the existence of the nanoclusters.

The zinc nanoclusters of the invention exhibit optical properties, particularly fluorescence, which are characteristic of this intermediate scale between molecule and nanoparticle.

### Stability of the Zinc Nanoclusters

The stability of the zinc nanoclusters was assessed by measuring their hydrodynamic diameter, using dynamic light scattering (173° angle, 530 nm laser, 25° C. temperature in a Malvern Nanosizer).

The analyses were performed on zinc nanoclusters obtained using the solution-phase protocol and the solid-phase protocol.

For the solution-phase protocol, the analyses were performed on 1x zinc nanocluster solutions having a zinc concentration of 16 μg/mL. Stability was assessed slightly more than 5 weeks after synthesis.

The hydrodynamic diameter of the zinc nanoclusters was still found to be 0.72±0.05 nm more than 5 weeks after synthesis, demonstrating their excellent stability.

For the solid-phase protocol, the analyses were performed on:
freeze-dried samples obtained from non-dialyzed liquid of zinc nanoclusters, having a zinc concentration of 1370 μg/mL (100x),
solutions reconstituted after freeze-drying said samples, to the same volume as was freeze dried.

The hydrodynamic diameter of the zinc nanoclusters from the freeze-dried samples was 1.73 nm after more than 5 weeks of storage under nitrogen.

After reconstitution of the samples to the same volume as was freeze-dried, the hydrodynamic diameter of the zinc nanoclusters in the reconstituted samples was 1.58 nm, further demonstrating their excellent stability.

### 2/Characterization of the Zinc and Selenium Nanoclusters

The zinc and selenium nanoclusters obtained in Example 1, section 2, whether via the solution-phase protocol or the solid-phase protocol, are characterized in terms of their structure, size, spectrophotometric properties, and stability.

### Structure of the Zinc and Selenium Nanoclusters

The zinc and selenium nanoclusters have a spherical shape. They are composed of a bimetal core of zinc and selenium covered with a mixed corona comprising histidine, acetate ions, and ascorbate ions. FIG. 8 is a schematic representation of a zinc and selenium nanocluster of the invention, which may also be designated by the formula “ZnSeNC@HisAcAsc”.

The presence of acetate ions on the surface of the bimetal zinc and selenium nanoclusters was demonstrated by high-performance liquid chromatography (HPLC), more specifically by reversed-phase chromatography.

The zinc and selenium nanoclusters in the dialyzed solution obtained in Example 1, section 2.1/“Solution-phase protocol”, are destroyed (total dissolution and a return of the nanoclusters structure to its various component elements) by a chemical process, namely dissolution in concentrated acid (HCl) then a concentrated base (NaOH), then are analyzed by reversed-phase chromatography with comparison to a sodium acetate control (retention of the component anions of the nanocluster on a cation exchange resin, separation, identification, and quantification (external calibration) of the acetate ions by conductometry with the aid of a suppressor).

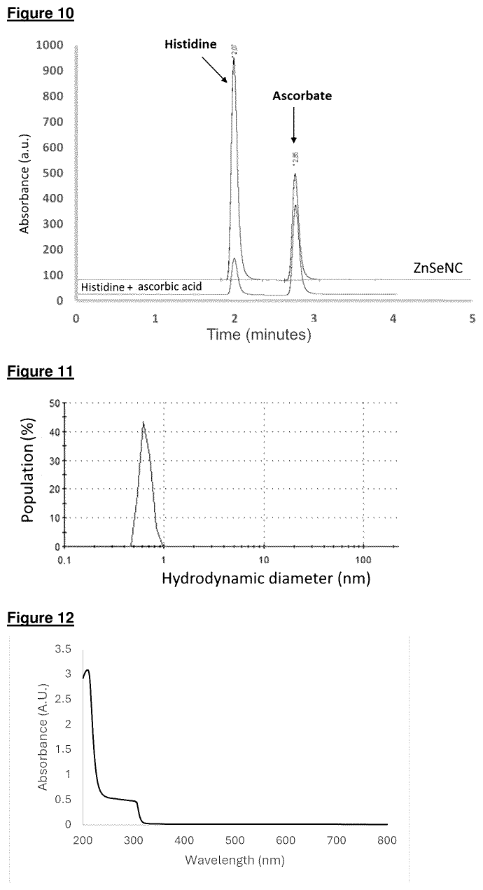

The results obtained are illustrated in the chromatogram in FIG. 9. The peak at 3.44 min associated with acetate ions (control, see lower plot) is found in the zinc and selenium nanoclusters (see upper plot), thus demonstrating the presence of acetate ions on the surface of the zinc and selenium bimetal core.

The presence of histidine and ascorbate ions on the surface of the zinc and selenium nanoclusters was also demonstrated by high-performance liquid chromatography, more specifically after purification of the nanoclusters in solution by size exclusion chromatography, in comparison to a histidine and ascorbic acid control.

The results obtained are illustrated in the chromatogram in FIG. 10.

The peak at 2.07 min associated with histidine (control, see lower plot) is found in the zinc and selenium nanoclusters (see upper plot), thus demonstrating the presence of histidine on the surface of the zinc and selenium bimetal core.

The peak at 2.85 min associated with ascorbate ions (control, see lower plot) is found in the zinc and selenium nanoclusters (see upper plot), thus demonstrating the presence of ascorbate ions on the surface of the zinc and selenium bimetal core.

### Size of the Zinc and Selenium Nanoclusters

The hydrodynamic diameters (Dh) of the zinc and selenium nanoclusters were evaluated by dynamic light scattering (FIG. 11) (angle 173°, 530 nm laser, 25° C. temperature in a Malvern Nanosizer).

The diameters of the zinc and selenium nanoclusters were evaluated immediately after their synthesis, for both the solution-phase and solid-phase protocols.

For the solution-phase protocol, the hydrodynamic diameters were evaluated more particularly on the non-dialyzed and non-freeze-dried solution of zinc and selenium nanoclusters that was obtained in section 2.1/of Example 1, which has a zinc concentration of 65 μg/mL (4x) and a selenium concentration of 0.1 μg/mL (0.1% Se).

For the solid-phase protocol, the hydrodynamic diameters were evaluated on the freeze-dried samples obtained in section 2.2/of Example 1, which has a zinc concentration of 1370 μg/mL (100x) and a selenium concentration of 16 μg/mL (1% Se).

It can be seen from FIG. 11 that the average hydrodynamic diameter of the zinc and selenium nanoclusters is 0.69±0.06 nm.

### Spectrophotometric Properties

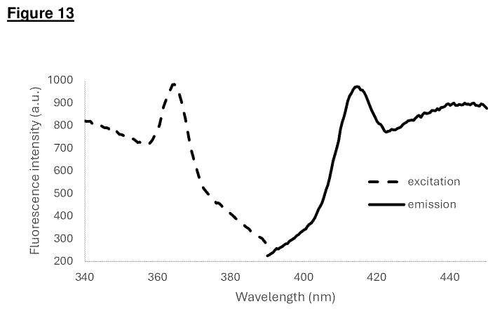

The spectrophotometric properties of the zinc and selenium nanoclusters were evaluated by UV-Visible spectroscopy (FIG. 12) and fluorescence spectroscopy (FIG. 13), immediately after their synthesis.

The UV-vis spectrum shows a shoulder at 300±15 nm (FIG. 12), confirming the existence of the nanoclusters.

Fluorescence of the zinc and selenium nanoclusters occurs with (FIG. 13) an excitation wavelength of 364±15 nm and an emission wavelength of 415±15 nm, also confirming the existence of the nanoclusters.

The zinc and selenium nanoclusters of the invention exhibit optical properties, particularly fluorescence, which are characteristic of this intermediate scale between molecule and nanoparticle.

### Stability of the Zinc and Selenium Nanoclusters

The stability of the zinc and selenium nanoclusters was assessed by measuring their hydrodynamic diameter using dynamic light scattering (173° angle, 530 nm laser, 25° C. temperature in a Malvern Nanosizer).

The analyses were performed on zinc and selenium nanoclusters obtained using the solution-phase protocol and the solid-phase protocol.

For the solution-phase protocol, the analyses were performed on the zinc and selenium nanocluster solutions obtained in section 2.1/of Example 1.

The hydrodynamic diameter of the zinc and selenium nanoclusters was found to be 0.69±0.06 nm more than 5 weeks after their synthesis, demonstrating their excellent stability.

For the solid-phase protocol, the analyses were performed on:
the freeze-dried samples obtained in item 2.2/of Example 1,
the solutions reconstituted after freeze-drying said samples, as obtained in item 2.2/of Example 1.

The hydrodynamic diameter of the zinc and selenium nanoclusters in the freeze-dried samples was 0.70 nm after more than 5 weeks of storage under nitrogen.

After reconstitution of the samples to the same volume as was freeze-dried, the hydrodynamic diameter of the zinc and selenium nanoclusters in the reconstituted samples was 0.70 nm, which once again demonstrates their excellent stability.

1-17. (canceled)

18. Zinc-based nanoclusters, comprising a zinc-based metal core covered over its entire surface with a mixed layer comprising histidine (His), acetate ions (Ac), and ascorbate ions (Asc), said metal core being composed of zinc (Zn) or a mixture of zinc and selenium (ZnSe), the zinc in said mixture ranging from 80 to 99.99% and the selenium in said mixture ranging from 0.01 to 20%, the percentages being percentages by weight relative to the total weight of the mixture,
wherein the zinc-based nanoclusters:
have a spherical shape,
have a hydrodynamic diameter ranging from 0.6 to 2.0 nm,
have a metal core diameter ranging from 0.5 to 1.5 nm,
exhibit a stability duration ranging from 5 to 20 weeks when the nanoclusters are in liquid form and are stored at a temperature of 4° C.,
exhibit a stability duration of at least 12 months, and preferably from 12 to 18 months, when the nanoclusters are in dry form and are stored at a temperature of 4° C. and under nitrogen, and
exhibit spectrophotometric properties, with a shoulder in the UV-Visible spectrum at 300±15 nm and a fluorescence spectrum with excitation wavelengths of 364±15 nm and emission wavelengths of 415±15 nm,
wherein the zinc-based nanoclusters having a metal core composed of Zn are referred to as having a formula ZnNC@HisAcAsc, and the zinc-based nanoclusters having a metal core composed of the ZnSe referred to as having a formula ZnSeNC@HisAcAsc.

19. The zinc-based nanoclusters according to claim 18, wherein the zinc nanoclusters comprise a metal core composed of Zn, referred to as having the formula ZnNC@HisAcAsc.

20. The zinc-based nanoclusters according to claim 18, wherein the zinc nanoclusters comprise a metal core composed of ZnSe, referred to as having the formula ZnSeNC@HisAcAsc.

21. The zinc-based nanoclusters according to claim 18, wherein the zinc-based nanoclusters are in liquid form or in dry form.

22. The zinc-based nanoclusters according to claim 18, wherein the zinc-based nanoclusters exhibit at least one of the following characteristics:
the zinc-based nanoclusters are able to cross the intestinal barrier,
the zinc-based nanoclusters exhibit good bioavailability,
the zinc-based nanoclusters are biocompatible,
the zinc-based nanoclusters are biodegradable,
the zinc-based nanoclusters are able to be freeze-dried,
the zinc-based nanoclusters are non-toxic to the human body, and
the zinc-based nanoclusters do not accumulate in organs such as the liver, spleen, kidneys, or lungs.

23. A method of preparing zinc-based nanoclusters according to claim 18, comprising the following steps:
reacting zinc (II) acetate dihydrate with histidine, or reacting zinc (II) acetate dehydrate and sodium selenite with histidine, in order to obtain a mixture of zinc (II) acetate dihydrate and histidine, or a mixture of zinc (II) acetate dihydrate, sodium selenite, and histidine, the molar ratio of histidine/zinc (II) acetate dihydrate or the molar ratio of histidine/(zinc (II) acetate dihydrate+sodium selenite) being greater than or equal to 10;
reacting the mixture of zinc (II) acetate dihydrate and histidine with ascorbic acid, or reacting the mixture of zinc (II) acetate dihydrate, sodium selenite, and histidine with ascorbic acid, in order to obtain a mixture of zinc (II) acetate dihydrate, histidine, and ascorbic acid, or a mixture of zinc (II) acetate dihydrate, sodium selenite, histidine, and ascorbic acid, the molar ratio of ascorbic acid/zinc (II) acetate dihydrate or the molar ratio of ascorbic acid/(zinc (II) acetate dihydrate+sodium selenite) being greater than or equal to 16;
collecting the zinc-based nanoclusters, more particularly collecting the zinc nanoclusters or zinc and selenium nanoclusters.

24. The method according to claim 23, wherein the method is carried out under inert gas and:
the zinc (II) acetate dihydrate, sodium selenite is in solution form and the histidine is in powder form,
a solution of zinc (II) acetate dihydrate and histidine, or a solution of zinc (II) acetate dihydrate, sodium selenite, and histidine, is prepared by adding histidine to the solution of zinc (II) acetate dihydrate, or to the solution of zinc (II) acetate dihydrate and sodium selenite,
the solution of zinc (II) acetate dihydrate and histidine, or the solution of zinc (II) acetate dihydrate, sodium selenite, and histidine, is adjusted to a pH value ranging from 11 to 13,
the ascorbic acid is in powder form,
a solution of zinc (II) acetate dihydrate, histidine, and ascorbic acid, or a solution of zinc (II) acetate dihydrate, sodium selenite, histidine, and ascorbic acid, is prepared by adding ascorbic acid to the solution of zinc (II) acetate dihydrate and histidine, or to the solution of zinc (II) acetate dihydrate, sodium selenite, and histidine, for which the pH has been adjusted to the above-mentioned values,
the solution of zinc (II) acetate dihydrate, histidine, and ascorbic acid, or the solution of zinc (II) acetate dihydrate, sodium selenite, histidine, and ascorbic acid, is stirred for 2 to 6 hours, and preferably 4 hours, at a temperature ranging from 35 to 45° C., and preferably 40° C.,
a solution comprising zinc-based nanoclusters is obtained at the end of the previous stirring step, namely a solution comprising zinc nanoclusters or zinc and selenium nanoclusters,
the solution comprising zinc-based nanoclusters is optionally dialyzed to obtain a purified solution of zinc-based nanoclusters, namely a purified solution of zinc nanoclusters or of zinc and selenium nanoclusters, and
the solution comprising the zinc-based nanoclusters, optionally dialyzed, is optionally freeze-dried to obtain a dry form of zinc-based nanoclusters, namely a dry form of the zinc nanoclusters or of zinc and selenium nanoclusters.

25. The method according to claim 24, wherein the method further comprises at least one characteristic selected from:
the inert gas is nitrogen,
the solution of zinc (II) acetate dihydrate is prepared by adding zinc (II) acetate dihydrate to filtered ultrapure water,
the solution of sodium selenite is prepared by adding sodium selenite to filtered ultrapure water,
the solution of zinc (II) acetate dihydrate has a concentration ranging from 0.5 to 5.0 mM,
the solution of sodium selenite has a concentration ranging from 0.07 to 140 mM,
the histidine concentration is higher than the concentration of the solution of zinc (II) acetate dihydrate,
the histidine concentration is higher than the concentration of the solution of sodium selenite,
the pH of the solution of zinc (II) acetate dihydrate and histidine, or of the solution of zinc (II) acetate dihydrate, sodium selenite, and histidine, is adjusted using sodium hydroxide,
the ascorbic acid concentration is equal to the histidine concentration,
the solution comprising the zinc nanoclusters, optionally dialyzed, has a zinc concentration ranging from 16 to 164 μg/mL,
the solution comprising the zinc and selenium nanoclusters, optionally dialyzed, has a zinc concentration ranging from 16 to 164 μg/mL and a selenium concentration ranging from 0.003 to 5.500 μg/mL, and
the solution comprising the zinc-based nanoclusters, optionally dialyzed, is freeze-dried to obtain a dry form of zinc-based nanoclusters, namely a dry form of zinc nanoclusters or of zinc and selenium nanoclusters.

26. The method according to claim 23, wherein:
the zinc (II) acetate dihydrate, sodium selenite, and histidine are each in powder form,
a powder mixture of zinc (II) acetate dihydrate and histidine, or a powder mixture of zinc (II) acetate dihydrate, sodium selenite, and histidine, is obtained by mixing together each of the powders of zinc (II) acetate dihydrate and histidine, or of zinc (II) acetate dihydrate, sodium selenite, and histidine,
the powder mixture of zinc (II) acetate dihydrate and histidine, or of zinc (II) acetate dihydrate, sodium selenite, and histidine, is ground until a powder mixture is obtained that is homogeneous in color and appearance,
the homogeneous powder mixture of zinc (II) acetate dihydrate and histidine, or of zinc (II) acetate dihydrate, sodium selenite, and histidine, is placed in a reactor,
the ascorbic acid is in powder form,
the ascorbic acid is added to the reactor comprising the homogeneous powder mixture of zinc (II) acetate dihydrate and histidine, or of zinc (II) acetate dihydrate, sodium selenite, and histidine,
the thus obtained powder mixture of zinc (II) acetate dihydrate, histidine, and ascorbic acid, or of zinc (II) acetate dihydrate, sodium selenite, histidine, and ascorbic acid, is stirred, then water is added dropwise into the reactor, said water being filtered ultrapure water,
the reactor is placed under inert gas and protected from light,
the mixture of zinc (II) acetate dihydrate, histidine, ascorbic acid, and water, or the mixture of zinc (II) acetate dihydrate, sodium selenite, histidine, ascorbic acid, and water, is left to stir in the reactor for 16 to 36 hours, and preferably 24 hours,
a liquid mixture comprising the zinc-based nanoclusters is obtained at the end of the previous stirring step, namely a liquid mixture of zinc nanoclusters or of zinc and selenium nanoclusters,
the liquid mixture comprising the zinc-based nanoclusters is optionally dialyzed to obtain a purified liquid mixture of zinc-based nanoclusters, namely a purified mixture of zinc nanoclusters or of zinc and selenium nanoclusters, and
the liquid mixture comprising the zinc-based nanoclusters, optionally dialyzed, is optionally freeze-dried to obtain a dry form of zinc-based nanoclusters, namely a dry form of zinc nanoclusters or of zinc and selenium nanoclusters.

27. The method according to claim 23, wherein the method further comprises at least one characteristic selected from:
the histidine concentration is greater than the zinc (II) acetate dihydrate concentration,
the histidine concentration is greater than the sodium selenite concentration,
the ascorbic acid concentration is equal to the histidine concentration,
the water added to the reactor is filtered ultrapure water,
the inert gas is nitrogen,
the liquid mixture comprising the zinc nanoclusters, optionally dialyzed, has a zinc concentration ranging from 1370 to 13700 μg/mL,
the liquid mixture comprising the zinc and selenium nanoclusters, optionally dialyzed, has a zinc concentration ranging from 1370 to 13700 μg/mL and a selenium concentration ranging from 0.16 to 3300 μg/mL, and
the liquid mixture comprising the zinc-based nanoclusters, optionally dialyzed, is freeze-dried to obtain a dry form of zinc-based nanoclusters, namely a dry form of zinc nanoclusters or of zinc and selenium nanoclusters.

28. The method according to claim 24, wherein the dry form of the zinc-based nanoclusters is stored under nitrogen and possible to store for a period of at least 12 months with no change in the stability of the zinc-based nanoclusters.

29. A medication comprising the zinc-based nanoclusters as defined in claim 18.

30. A method of combating zinc deficiencies in a subject in need thereof, comprising administering to said subject an effective amount of the zinc-based nanoclusters as defined in claim 18.

31. A method of combating zinc and selenium deficiencies in a subject in need thereof, comprising administering to said subject an effective amount of the zinc-based nanoclusters as defined in claim 18.

32. A composition comprising the zinc-based nanoclusters as defined in claim 18.

33. The composition according to claim 32, wherein the composition is a medication, a dietary supplement, or a food composition.

34. The composition according to claim 32, wherein the composition is in a form suitable for oral administration.
